(19) United States
(12) Patent Application Publication
(10) Pub. No.: US 2004/0265347 A1
(43) Pub. Date: Dec. 30, 2004

(54) Cosmetic composition comprising a sebum-absorbing powder and a powder with a low critical surface energy

(76) Inventors: Frederic Auguste, Chevilly-Larue (FR); Anke Hadasch, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/833,166

(22) Filed: Apr. 28, 2004

Related U.S. Application Data
(60) Provisional application No. 60/467,581, filed on May 5, 2003.

(30) Foreign Application Priority Data
Apr. 28, 2003 (FR) 03 05174

(51) Int. Cl. A61K 7/00

(52) U.S. Cl. 424/401

(57) ABSTRACT

Disclosed herein is a cosmetic composition comprising at least one sebum-absorbing powder having a sebum uptake, at least one second powder having a critical surface energy of less than or equal to 24 mN/m, and a liquid binder phase comprising at least one non-volatile oil.

The composition may make it possible to obtain a skin makeup with good cosmetic properties over time, such as a good rub resistance, transfer-free properties, good uniformity, maintenance of the initial color of the makeup, and the absence of sheen (i.e., resistance of the matte effect).

This application claims benefit of U.S. Provisional Application No. 60/467,581, filed May 5, 2003.

Disclosed herein is a cosmetic composition comprising a mixture of powders. Also disclosed herein is a process for making up and/or caring for keratin materials, such as human skin, lips, nails, and hair, comprising applying the composition to the keratin materials.

The composition disclosed herein may be a makeup and/or care composition for keratin materials, for example a skin makeup composition, such as a foundation, an eyeshadow, a makeup rouge, a concealer product, a face and body powder, and a body makeup product.

Makeup powders generally comprise, firstly, a pulverulent phase that may comprise pigments and fillers, and, secondly, a fatty phase that acts as a binder comprising fatty substances (i.e., a binder phase), intended to give the finished product a certain amount of density, to give the makeup product softness and an emollient nature, and to promote its adhesion to the skin.

After makeup is applied to the skin, the sebum excreted by the skin over time may modify the cosmetic properties of the makeup. For example, sebum may not promote the adhesion of the makeup to the skin. The makeup may have a tendency to transfer onto surfaces or fabrics placed in contact with the made-up skin, resulting in a loss of makeup remaining on the skin. In addition, the sebum-impregnated makeup may have a tendency to migrate more easily into the folds or wrinkles of the face, making the makeup deposit non-uniform. Furthermore, sebum may make the makeup more sensitive to rubbing, for example to rubbing against fabrics or the fingers, causing removal of the makeup over time. The poor staying power of the makeup in the course of the day may make it necessary for the user to freshen the application of the makeup in order to maintain a uniform makeup effect.

Moreover, sebum has a tendency to modify the color of the makeup deposited on the skin: for example, the color may become darker. This change in color may go against the desired colored aesthetic effect, since the visual appearance of the makeup no longer corresponds to the initial color of the composition chosen. The makeup may also become shinier; however, this shiny appearance is not desired for a foundation, since consumers prefer a matte makeup that maintains this matte effect throughout the day.

It is known practice from WO 97/04737 to use sebum-absorbing powders as a mixture with fillers and/or pigments in makeup powder compositions. However, the fillers and pigments may also become impregnated with sebum, and the adsorption of sebum does not take place selectively by the sebum-absorbing powders. The drawbacks caused by the excretion of sebum over time, for example the change in color and the shiny appearance of the makeup, thus remain for these compositions.

One aim of the present inventors was thus to provide a makeup composition that can efficiently absorb sebum and thus give a makeup with good cosmetic properties over time, such as good resistance to rubbing, transfer-resistant properties, good homogeneity, maintenance of the initial color of the makeup, and absence -of sheen (i.e., resistance of the matte effect).

The inventors have discovered that such a composition may be obtained by using at least one sebum-absorbing powder combined with at least one second powder having a specified critical surface energy. This powder with a specified critical surface energy may not become impregnated with sebum when the makeup is deposited on the skin, and thus may prevent sebum excreted over time from modifying the cosmetic properties of the makeup.

For example, one embodiment disclosed herein is a cosmetic composition comprising a pulverulent phase comprising at least one sebum-absorbing powder having the ability to uptake sebum, at least one second powder having a critical surface energy of less than or equal to 24 mN/m, and a liquid binder phase.

Another embodiment is a non-therapeutic cosmetic process for making up and/or caring for keratin materials, comprising applying to the keratin materials a composition as defined above. The keratin materials may, for example, be human skin.

Yet another embodiment is the use of a composition as defined above to obtain a non-shiny, uniform, transfer-resistant, and/or rub-resistant makeup result on the skin and/or a makeup result that shows maintenance of the initial color.

A further embodiment disclosed herein is the use of at least one sebum-absorbing powder having the ability to uptake sebum, and of at least one second powder having a critical surface energy of less than or equal to 24 mN/m, in a cosmetic composition to obtain a non-shiny, uniform, transfer-resistant, and/or rub-resistant makeup result on the skin and/or a makeup result that shows maintenance of the initial color.

As disclosed above, the composition disclosed herein comprises at least one sebum-absorbing powder having the ability to uptake sebum. In certain embodiments, the sebum uptake of the powder is greater than or equal to 1 ml/g, such as a sebum uptake ranging from 1 ml/g to 20 ml/g, or ranging from 1 ml/g to 15 ml/g. For example, the at least one sebum-absorbing powder may have a sebum uptake of greater than or equal to 1.5 ml/g, such as a sebum uptake ranging from 1.5 ml/g to 20 ml/g, or ranging from 1.5 ml/g to 15 ml/g. As a further example, the sebum uptake may be greater than or equal to 2 ml/g, such as a sebum uptake ranging from 2 ml/g to 20 ml/g, or ranging from 2 ml/g to 15 ml/g.

As used herein, the term “sebum-absorbing powder” means a powder capable of absorbing and/or adsorbing sebum.

The sebum uptake corresponds to the amount of sebum absorbed and/or adsorbed by the powder. It may be measured according to the Wet Point method described below.

In certain embodiments, the at least one sebum-absorbing powder may have a BET specific surface area of greater than or equal to 300 m²/g, such as greater than 500 m²/g, for example greater than 600 m²/g, and for example less than 1500 m²/g.

The “BET specific surface area” is determined according to the BET (Brunauer-Emmet-Teller) method described in “The Journal of the American Chemical Society”, Vol. 60, page 309, February 1938, and corresponding to ISO international standard 5794/1 (appendix D). The BET specific surface area corresponds to the total specific surface area (thus including micropores) of the powder.

The at least one sebum-absorbing powder may be a powder chosen from mineral powders and organic powders. It may be chosen from silica; polyamide powders (NYLON®); acrylic polymer powders, such as polymethyl methacrylate powder, polymethyl methacrylate/ethylene glycol dimethacrylate powder, polyallyl methacrylate/ethylene glycol dimethacrylate powder, and ethylene glycol dimethacrylate/lauryl methacrylate copolymer powder; and silicone elastomer powders, obtained for example by polymerization of organopolysiloxane comprising at least two hydrogen atoms each linked to a silicon atom, and of an organopolysiloxane comprising at least two ethylenically unsaturated groups (such as two vinyl groups) in the presence of a platinum catalyst.

The at least one sebum-absorbing powder may be a powder coated with at least one hydrophobic treatment agent.

The at least one hydrophobic treatment agent may be chosen from fatty acids, for instance stearic acid; metal soaps, for instance aluminium dimyristate; the aluminium salt of hydrogenated tallow glutamate; amino acids; N-acylamino acids and salts thereof; lecithin; isopropyl triisostearyl titanate; and mixtures thereof.

The N-acylamino acids may comprise an acyl group comprising 8 to 22 carbon atoms, for instance a group chosen from 2-ethylhexanoyl, caproyl, lauroyl, myristoyl, palmitoyl, stearoyl, and cocoyl groups. The salts of these compounds may be salts chosen from aluminium, magnesium, calcium, zirconium, zinc, sodium and potassium salts. The amino acid may be chosen, for example, from lysine, glutamic acid, and alanine.

As used herein, the term “alkyl” mentioned in the compounds mentioned above may denote an alkyl group comprising 1 to 30 carbon atoms, such as 5 to 16 carbon atoms.

Silica powders that may be mentioned include:

the hollow silica microspheres sold under the name Silica Beads SB-700 by the company Myoshi; SUNSPHERE® H51 and SUNSPHERE® H33 by the company Asahi Glass; and

the amorphous silica microspheres coated with polydimethylsiloxane, sold under the name SA SUNSPHERE® H33 and SA SUNSPHERE® H53 by the company Asahi Glass.

Acrylic polymer powders that may be mentioned include:

the polymethyl methacrylate powders sold under the name COVABEAD® LH85 by the company Wackherr;

the polymethyl methacrylate/ethylene glycol dimethacrylate powders sold under the name Dow Corning 5640 MICROSPONGE® Skin Oil Adsorber by the company Dow Corning; GANZPEARL® GMP-0820 by the company Ganz Chemical;

the polyallyl methacrylate/ethylene glycol dimethacrylate powders sold under the name POLY-PORE® L200 and POLY-PORE® E200 by the company Amcol; and

the ethylene glycol dimethacrylate/lauryl methacrylate copolymer powders sold under the name POLYTRAP® 6603 by the company Dow Corning.

Nylon powders that may be mentioned include the nylon powder sold under the name ORGASOL® 4000 by the company Atochem.

Silicone elastomer powders that may be mentioned include the powders sold under the names TREFIL® Powder E-505C and TREFIL® powder E-506C by the company Dow Corning.

One sebum-absorbing powder that may be used is silica powder, having the characteristics described above, such as having a sebum uptake of greater than or equal to 2 ml/g, for example a sebum uptake ranging from 2 ml/g to 20 ml/g. Such a silica powder is sold under the name SUNSPHERE® H33 by the company Asahi Glass.

The at least one sebum-absorbing powder may be present in the composition disclosed herein in a content ranging from 1% to 98% by weight, such as ranging from 1% to 80% by weight, 5% to 80% by weight, ranging from 1% to 60% by weight, ranging from 5% to 60% by weight, ranging from 1% to 35% by weight, or ranging from 1% to 15% by weight, relative to the total weight of the composition.

The composition disclosed herein also comprises at least one additional powder, referred to as the second powder, having a critical surface energy of less than or equal to 24 mN/m (such as ranging from 10 to 24 mN/n) and which may, for example, be less than or equal to 20 mN/m (such as ranging from 10 to 20 mN/m).

The critical surface energy of a powder may be measured according to the method described below.

The at least one second powder may be a powder coated with a compound chosen from organofluorine compounds and silicone compounds.

The powder (intended to be coated) may be chosen from the pigments, nacres, and fillers usually used in cosmetic compositions, such as those described below in the description of the additional powders.

The organofluorine compound coating the powder may be a compound chosen from perfluoroalkyl phosphate and polyhexafluoropropylene oxide.

Powders coated with perfluoroalkyl phosphate are described for example in U.S. Pat. No. 3,632,744 and Japanese Patent Nos. JP-A-04 330 007 and JP-A-2001-316 223.

The perfluoroalkyl phosphate may be chosen from those of formula (I)

[CₘF₂ₘ₊₁CₙH₂ₙO]_{y}PO(OM)_{3-y}

wherein m is an integer ranging from 1 to 21; n is an integer from 1 to 14; y is an integer chosen from 1, 2, and 3; M is an entity chosen from hydrogen, alkali metal cations, ammonium groups, and ammonium groups substituted with 1 to 3 C₁-C₈ alkyl radicals.

According to another embodiment, the at least one second powder may be a powder coated with a silicone compound, for instance compounds chosen from methicones, dimethicones, polyorganosiloxanes comprising perfluoroalkyls, polyorganosiloxanes comprising perfluoropolyethers, and perfluoroalkylsilanes.

As examples of the coated powder having a critical surface energy as defined above, mention may be made of:

the powders coated with perfluoroalkyl phosphate and with butyl acrylate/perfluoro(C₆-C₁₄)alkyl ethyl acrylate/mercaptopropyldimethicone copolymer sold under the names Novatech® NFP Double Treated Talc, Novatech® NFP Double Treated Sericite, Novatech® NFP Double Treated Yellow Iron Oxide, Novatech® NFP Double Treated Red Iron Oxide, Novatech® NFP Double Treated Black Iron Oxide, and Novatech® NFP Double Treated Titanium Dioxide by the company Daikin;

the powders coated with perfluoroalkyl phosphate, with butyl acrylate/perfluoro(C₆-C₁₄)alkylethyl acrylate/mercaptopropyldimethicone copolymer, and with hydroxyethyl methacrylate/perfluoro(C₆-C₁₄)alkylethyl acrylate copolymer sold under the names Novatech® NFP Triple Treated Talc, Novatech® NFP Triple Treated Sericite, Novatech® NFP Triple Treated Yellow Iron Oxide, Novatech® NFP Triple Treated Red Iron Oxide, Novatech® NFP Triple Treated Black Iron Oxide and Novatech® NFP Triple Treated Titanium Dioxide by the company Daikin.

The compound chosen from organofluorine compounds and silicone compounds may be present in an amount ranging from 0.01% to 60% by weight, such as ranging from 0.05% to 40% by weight, or ranging from 0.1% to 40% by weight, relative to the total weight of the coated powder.

The at least one second powder as described above may be present in the composition disclosed herein in a content ranging from 0.1% to 95% by weight, such as ranging from 0.5% to 90% by weight, ranging from 1% to 90% by weight, or ranging from 5% to 80% by weight, relative to the total weight of the composition.

The binder phase of the composition may promote good dispersion of the powders. This binder phase may be a fatty phase that is liquid at room temperature (25° C.), such as a non-volatile liquid fatty phase.

The liquid fatty phase may be present in an amount ranging from 0.1% to 90% by weight, such as ranging from 0.1% to 75% by weight, ranging from 1% to 50% by weight, or ranging from 1% to 30% by weight, relative to the total weight of the composition.

The liquid fatty phase may comprise at least one non-volatile oil.

As used herein, the term “non-volatile oil” means an oil capable of remaining on the skin at room temperature (25° C.) and atmospheric pressure for at least one hour, for example an oil having a non-zero vapor pressure at room temperature (25° C.) and atmospheric pressure of less than or equal to 0.01 mmHg (1.33 Pa).

In certain embodiments, the non-volatile liquid fatty phase may comprise at least one non-volatile oil with a viscosity, measured at 25° C., of greater than or equal to 5×10⁻² Pa.s (50 cps), such as ranging from 5×10⁻² Pa.s to 40 Pa.s (40 000 cps), or for example a viscosity greater than or equal to 9×10⁻² Pa.s (90 cps), such as ranging from 9×10⁻² Pa.s to 40 Pa.s.

The viscosity is measured at 25° C.±0.5° C. using a Haake RS75 controlled-stress rheometer from the company Thermo Rheo, equipped with a spindle of cone/plate geometry with a diameter ranging from 2 cm to 6 cm and an angle ranging from 1° to 2°, the choice of the spindle depending on the viscosity to be measured (i.e., the more fluid the oil, the greater the diameter of the chosen cone and the smaller the angle). The measurement is performed by applying on the oil sample a logarithmic shear stress gradient τ ranging from 0.2 Pa to 1000 Pa for a duration of 20 minutes. The rheogram representing the change in viscosity as a function of the change in shear ε′ is then plotted. The rheogram shows a plateau at low shear rate values (known as a Newtonian plateau). This plateau corresponds to a stable viscosity value, which is the viscosity of the oil thus determined.

The non-volatile liquid fatty phase may comprise at least one non-volatile oil.

In certain embodiments, the at least one non-volatile oil has Hansen solubility parameters δ^{L}_{d}, δ^{L}ₚ, and δ^{L}ₕ, expressed in J^{1/2}.cm^{-3/2}, such that

Δδ=\sqrt{4(16.4−δ_{d}^{L})²+(0.9−δₚ^{L})²+(4.2−δₕ^{L})²}

wherein Δδ ranges from 2 to 20, such as from 4 to 16 or from 6 to 16.

δ^{L}_{d} is the dispersive component of the solubility parameter of the at least one non-volatile oil,

δ^{L}ₚ is the polar component of the solubility parameter of the at least one non-volatile oil,

δ^{L}ₕ is the component of the specific bonds (hydrogen bonds) of the solubility parameter of the at least one non-volatile oil.

In certain embodiments, δ^{L}_{d} is less than or equal to 17, such as less than or equal to 14.

The solubility parameters are the Hansen solubility parameters as defined in the article by C. M. Hansen “The three dimensional solubility parameters”, J. Paint Technol. 39, 105 (1967) and in the book “Handbook of Solubility Parameters”, Allan F. Barton, CRC Press, 1991.

The at least one non-volatile oil as described above may be chosen from triisoarachidyl citrate, polyvinylpyrrolidone/hexadecene with a weight-average molecular weight ranging from 5,000 to 9,000 (such as the product sold under the name ANTARON® V-216 by the company ISP), 2-butyloctyl trimellitate, triisostearyl citrate, tridecyl trimellitate, polyglyceryl-2 triisostearate, pentaerythrityl tetrakis(2-ethylhexanoate), triisodecyl trimellitate, diisostearyl malate, tris(2-ethylhexyl) trimellitate, 2-octyldodecanol, octyl hydroxystearate, polybutylene with a-weight-average molecular weight ranging from 800 to 1,200 (such as the product sold under the name INDOPOL® H-100 by the company Amoco), isostearyl lactate, propylene glycol monoisostearate, polyglyceryl-2 diisostearate, castor oil, dipropylene glycol dibenzoate, oxyethylenated (7 EO) glyceryl triacetate, polyglyceryl-3 diisostearate and the polymethyltrifluoropropyldimethylsiloxane of formula (II):

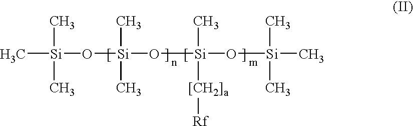

in which

n is an integer ranging from 5 to 90, for example ranging from 30 to 80 or ranging from 50 to 80;

m is an integer ranging from 1 to 150, for example ranging from 1 to 80 or ranging from 1 to 40;

a is an integer ranging from 0 to 5; and

Rf is a perfluoroalkyl radical comprising 1 to 8 carbon atoms.

Compounds of formula (II) that may be mentioned include those sold under the names X22-819, X22-820, X22-821, and X22-822 by the company Shin-Etsu.

One non-volatile oil that may be mentioned is the polymethyltrifluoropropyl-dimethylsiloxane of formula (II) defined above. For example, this non-volatile oil may be used with the sebum-absorbing silica powder described above.

The at least one non-volatile oil as defined above may be present in the composition disclosed herein in an amount ranging from 0.1% to 90% by weight, for example from 0.1% to 75% by weight, from 1% to 50% by weight, or from 1% to 30% by weight, relative to the total weight of the composition.

According to one embodiment of the composition disclosed herein, the liquid fatty phase may comprise 100% by weight of the at least one non-volatile oil as defined above.

The liquid fatty phase of the composition disclosed herein may comprise at least one additional non-volatile oil that is different from the at least one non-volatile oil described above. In one embodiment, the at least one additional non-volatile oil has Hansen parameters such that Δδ is less than 2 or greater than 16 and/or a viscosity of less than 5×10⁻² Pa.s.

The at least one additional non-volatile oil may be an oil chosen from hydrocarbon-based oils, silicone oils, and fluoro oils.

As used herein, the term “hydrocarbon-based oil” means an oil formed from or comprising carbon and hydrogen atoms, optionally formed from or comprising oxygen and nitrogen atoms, and not comprising silicon or fluorine atoms. A hydrocarbon oil may comprise at least one group chosen from alcohol, ester, ether, carboxylic acid, amine, and amide groups.

Non-volatile oils that may be mentioned as-the additional non-volatile oils useful herein include mink oil; turtle oil; soybean oil; grapeseed oil; sesame seed oil; maize oil; rapeseed oil; sunflower oil; cotton seed oil; avocado oil; olive oil; jojoba oil; groundnut oil; hydrocarbon oils such as liquid paraffins, squalane, and petroleum jelly; fatty esters such as isopropyl myristate, isopropyl palmitate, butyl stearate, hexyl laurate, isononyl isononate, 2-ethylhexyl palmitate, 2-hexyldecyl laurate, 2-octyldecyl palmitate, 2-octyldodecyl myristate, 2-octyldodecyl lactate, 2-diethylhexyl succinate, diisostearyl malate, glyceryl triisostearate, and diglyceryl triisostearate; perfluoro oils; higher fatty acids such as myristic acid, palmitic acid, stearic acid, behenic acid, oleic acid, linoleic acid, linolenic acid, and isostearic acid; higher fatty alcohols such as oleyl alcohol; and mixtures thereof.

When the composition disclosed herein comprises the at least one additional non-volatile oil, this at least one additional non-volatile oil may be present in an amount by volume of less than 0.5×(sebum uptake of the sebum-absorbing powder)×(weight amount of sebum-absorbing powder present in the composition), for example less than 0.4×(sebum uptake of the sebum-absorbing powder)×(weight amount of sebum-absorbing powder present in the composition) and for example less than 0.3×(sebum uptake of the sebum-absorbing powder)×(weight amount of sebum-absorbing powder present in the composition).

Non-volatile oils that may also be used include perfluoropolyether oils such as the perfluoro polymethyl isopropyl ether with a weight-average molecular weight of 1500, sold under the name FOMBLIN® HC04 by the company Ausimont.

The non-volatile liquid fatty phase comprising the at least one non-volatile oil, and optionally the at least one additional non-volatile oil, may be present in the composition disclosed herein in an amount ranging from 0.1% to 90% by weight, for example ranging from 0.1% to 75% by weight, ranging from 1% to 50% by weight, or ranging from 1% to 30% by weight, relative to the total weight of the composition.

The liquid fatty phase may also comprise at least one volatile oil.

As used herein, the term “volatile oil” means an oil capable of evaporating from the skin in less than one hour at room temperature and atmospheric pressure. This at least one volatile oil may have a vapor pressure, at room temperature (25° C.) and atmospheric pressure (760 mmHg) ranging from 0.01 to 300 mmHg (1.33 Pa to 40,000 Pa), for example ranging from 0.05 to 300 mmHg (6.65 Pa to 40,000 Pa).

The at least one volatile oil may be chosen from carbon-based oils; hydrocarbon-based oils; fluoro oils; silicone oils of mineral, animal, plant and synthetic origin; and mixtures thereof.

Volatile oils that may be mentioned include linear or cyclic silicone oils with a viscosity at room temperature of less than 8 mm²/s, for example volatile oils comprising 2 to 7 silicon atoms, these silicones optionally comprising alkyl or. alkoxy groups comprising 1 to 10 carbon atoms. As volatile silicone oils that may be used according to the present disclosure, mention may be made of octamethylcyclotetrasiloxane, decamethyl-cyclopentasiloxane, dodecamethylcyclohexasiloxane, heptamethylhexyltrisiloxane, heptamethyloctyltrisiloxane, hexamethyid isiloxane, octamethyltrisiloxane, decamethyltetrasiloxane, dodecamethylpentasiloxane, and mixtures thereof.

Other volatile oils that may be mentioned include hydrocarbon-based volatile oils comprising 8 to 16 carbon atoms and mixtures thereof, such as C₈-C₁₆ branched alkanes, for instance C₈-C₁₆ isoalkanes (also known as isoparaffins), isododecane, isodecane, isohexadecane, for example the oils sold under the trade names ISOPAR® and PERMETHYL®, C₈-C₁₆ branched esters, for instance isohexyl neopentanoate, and mixtures thereof. Isododecane may be used.

The at least one volatile oil may be present in an amount ranging from 0.1% to 90% by weight, for example ranging from 0.1% to 75% by weight or ranging from 1% to 50% by weight, relative to the total weight of the composition.

The composition disclosed herein may comprise an aqueous phase comprising water. The water may be chosen from floral waters such as cornflower water; mineral waters such as Vittel, Lucas, and La Roche Posay; and spring waters.

The aqueous phase may also comprise solvents other-than water, such as, for example, primary alcohols such as ethanol and isopropanol; glycols such as glycerol, propylene glycol, butylene glycol, dipropylene glycol, and diethylene glycol; glycol ethers such as mono-, di- and tripropylene glycol (C₁-C₄)alkyl ethers; mono-, di- and triethylene glycol; and mixtures thereof.

The aqueous phase may also comprise stabilizers, for example sodium chloride, magnesium dichloride, and magnesium sulphate.

The aqueous phase may also comprise any water-soluble or water-dispersible compound that is compatible with an aqueous phase, such as gelling agents, film-forming polymers, thickeners, surfactants, and mixtures thereof.

In certain embodiments, the aqueous phase, for example water, may be present in the composition disclosed herein in an amount ranging from 0.1% to 60% by weight, for example ranging from 5% to 50% by weight, relative to the total weight of the composition.

According to one embodiment, the binder phase may comprise an aqueous phase as described above.

According to another embodiment, the composition may be an anhydrous composition, i.e., a composition comprising less than 2% by weight of water, or less than 0.5% of water, the water not being added during the preparation of the composition but corresponding to the residual water provided by the mixed ingredients.

The pulverulent phase of the composition disclosed herein may also comprise at least one additional powder other than the at least one sebum-absorbing powder and the at least one second powders having a critical surface energy of less than or equal to 24 mN/m described above. This at least one additional powder may thus have a sebum uptake of less than 1 ml/g and a critical surface energy of greater than 24 mN/m.

The at least one additional powder may. be chosen from pigments, nacres, and fillers.

As used herein, the term “pigments” should be understood as meaning white or colored, mineral or organic particles of any form, which are insoluble in the physiological medium and are intended to color the composition.

As used herein, the term “nacres” should be understood as meaning iridescent particles of any form, which may be produced by certain molluscs in their shell, or which may be synthesized.

The pigments may be present in the composition in an amount ranging from 0.01% to 30% by weight, for example ranging from 1% to 20% by weight, relative to the weight of the composition.

The pigments may be white or colored, and mineral or organic. Among the mineral pigments that may be mentioned are titanium dioxide, optionally surface-treated, zirconium oxide, cerium oxide, zinc oxide, iron oxide (black, yellow, and red), chromium oxide, manganese violet, ultramarine blue, chromium hydrate, ferric blue, and metallic powders such as aluminium powder and copper powder.

Among the organic pigments that may be mentioned are carbon black, D & C pigments, and cochineal carmine; barium, strontium, calcium, and aluminium lakes.

The nacres may be present in the composition in an amount ranging from 0.01% to 50% by weight, for example ranging from 0.1% to 30% by weight, relative to the total weight of the composition.

The nacreous pigments may be chosen from white nacreous pigments such as mica coated with titanium and mica coated with bismuth oxychloride, colored nacreous pigments such as titanium mica coated with iron oxides, titanium mica coated with, for example, ferric blue and chromium oxide, titanium mica coated with an organic pigment of the abovementioned type, and also nacreous pigments based on bismuth oxychloride.

As used herein, the term “fillers” should be understood as meaning colorless or white, mineral or synthetic particles of any shape, which are insoluble in the medium of the composition irrespective of the temperature at which the composition is manufactured.

The fillers may be mineral or organic and of any shape, for example in a form chosen from platelet, spherical, and oblong, irrespective of the crystallographic shape (for example leaflet, cubic, hexagonal, orthorhombic, etc.). Mention may be made of talc, mica, silica, kaolin, powders of polyamide, for instance NYLON®, of poly-β-alanine and of polyethylene, lauroyllysine, starch, boron nitride, hollow polymer microspheres such as those of polyvinylidene chloride/acrylonitrile, for instance EXPANCEL® (Nobel Industrie), acrylic acid copolymers, precipitated calcium carbonate, magnesium carbonate and magnesium hydrocarbonate, hydroxyapatite, hollow silica microspheres, glass or ceramic microcapsules, metal soaps derived from organic carboxylic acids comprising 8 to 22 carbon atoms, such as 12 to 18 carbon atoms, for example zinc stearate, magnesium stearate, lithium stearate, zinc laurate, and magnesium myristate.

The fillers may be present in the composition in an amount ranging from 0.01% to 98.9% by weight, for example ranging from 0.1% to 85% by weight, relative to the total weight of the composition.

The at least one additional powder may be present in the composition in an amount ranging from 0.01% to 98.9% by weight, for example ranging from 0.1% to 85% by weight or ranging from 1% to 70% by weight, relative to the total weight of the composition.

The composition may comprise at least one other common cosmetic ingredient, which may be chosen, for example, from antioxidants, fragrances, preserving agents, neutralizers, surfactants, waxes, sunscreens, vitamins, moisturizers, self-tanning compounds, and antiwrinkle active agents.

Needless to say, a person skilled in the art will take care to select this or these optional additional compound(s), and/or the amount thereof, such that the advantageous properties of the composition disclosed herein are not, or are not substantially, adversely affected by the envisaged addition.

The composition disclosed herein may be in a form chosen from compact powders, pressed powders, cast powders, and free powders. It may also be in a form chosen from gels, water-in-oil emulsions, oil-in-water emulsions, multiple emulsions, milks, and pastes.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contain certain errors necessarily resulting from the standard deviation found in their-respective testing measurements.

Method for Measuring the Sebum Uptake of a Powder:

The sebum uptake of a powder may be measured according to the method for determining the oil uptake of a powder described in standard NF T 30-022. It corresponds to the amount of sebum adsorbed onto the available surface of the powder by measuring the Wet Point.

An amount m (in grams) of powder, ranging from 0.5 g to 5 g (the amount, depends on the density of the powder), is placed on a glass plate, followed by dropwise addition of artificial sebum having the following composition:

| | |
|---|---|
| triolein | 29% |
| oleic acid | 28.5% |
| oleyl oleate | 18.5% |
| squalene | 14% |
| cholesterol | 7% |
| cholesteryl palmitate | 3% |

After adding 4 to 5 drops of artificial sebum, the artificial sebum is incorporated into the powder using a spatula and the addition of artificial sebum is continued until conglomerates of artificial sebum and of powder are formed. At this point, the artificial sebum is added one drop at a time and the mixture is then triturated with the spatula. The addition of artificial sebum is stopped when a smooth, firm paste is obtained. This paste should be able to be spread out on the glass plate without cracking or forming lumps. The volume Vs (expressed in ml) of artificial sebum used is then noted.

The sebum uptake corresponds to the ratio Vs/m.

Method for Measuring the Critical Surface Energy of a Powder:

The powder is compacted using a pelletizer, such as the Manual Hydraulic Press manufactured by Perkin-Elmer (used to prepare samples analysed by infrared spectroscopy). The compacting pressure is 10 tonnes. The pellet of compacted powder has a diameter of about 10 mm and a thickness of about 1 mm.

The pellets of compacted powder are placed on the sample holder of a contact-angle tensiometer such as the tensiometer DAT 1100 sold by the company Fibro (Sweden).

The contact angle measurements are performed at 27±1° C. with the following three solvents: water, diiodomethane, and formamide.

For each of the solvents, one drop of solvent, with a volume ranging from 2 to 10 microliters, is placed on the powder pellet. The optical system and image analysis system of the tensiometer allow the change over time of the spreading of the drop to be monitored. The contact angle of the liquid on the compacted powder corresponds to the angle between the powder/liquid contact surface and the tangent to the drop passing through the point at which the drop, the air and the surface meet.

The contact angle-value used for the determination of the critical surface energy is the mean of the contact angles measured on 5 to 10 powder pellets, the angle values being taken after a contact time between the drop of solvent/powder pellet equal to 1 second.

For the determination of the critical surface energy, the change of the cosine of the contact angle as a function of the surface tension of the associated solvent is plotted on a graph, for the 3 test solvents.

The critical surface energy of the powder then corresponds to the surface tension such that the cosine of the contact angle is equal to 1 when the change in contact angle as a function of the surface tension is linearly extrapolated.

### EXAMPLE 1

A makeup powder having the composition below was prepared:

| | |
|---|---|
| Talc coated with perfluoroalkyl phosphate (NOVATECH ® | 35.4 g |
| NFP Talc-N2 from Daikin) | |
| Coated sericite (NOVATECH ® NFP Triple Treated Sericite | 30 g |
| from Daikin) | |
| Porous silica (SUNSPHERE ® H33 from Asahi Glass) | 10 g |
| Yellow iron oxide | 5 g |
| Brown iron oxide | 3 g |
| Ultramarine blue | 3.7 g |
| Titanium dioxide | 2.9 g |
| Fluorosilicone* (X22-819 from Shin-Etsu) | 10 g |

*Polymethyltrifluoropropyldimethylsiloxane

After this powder was applied to the skin, a makeup with a good ability to absorb the sebum excreted by the skin throughout the day, while remaining uniform, non-shiny and maintaining its initial color, was obtained.

### EXAMPLE 2

A foundation (W/O emulsion) having the composition below was prepared:

| | |
|---|---|
| Fluorosilicone* (X22-819 from Shin-Etsu) | 3.9 g |
| Cyclopentasiloxane | 15.5 g |
| Polydimethylsiloxane | 2.0 g |
| Bentonite | 2.5 g |
| 40/30/30 mixture of cetyldimethicone copolyol, polyglyceryl- | 1.7 g |
| 4 isostearate and hexyl laurate (ABIL ® WE 09 from | |
| Goldschmidt) | |
| Coated yellow iron oxide (NOVATECH ® NFP Double Treated | 1.0 g |
| Yellow Iron Oxide from Daikin) | |
| Coated brown iron oxide (NOVATECH ® NFP Double Treated | 0.4 g |
| Red Iron Oxide from Daikin) | |
| Coated black iron oxide (NOVATECH ® NFP Double Treated | 0.1 g |
| Black Iron Oxide from Daikin) | |
| Coated titanium dioxide (NOVATECH ® NFP Double Treated | 3.9 g |
| Titanium Dioxide from Daikin) | |
| Porous silica (SUNSPHERE ® H51 from Asahi Glass) | 4.0 g |
| Coated sericite (NOVATECH ® NFP Triple Treated Sericite | 6.0 g |
| from Daikin) | |
| Water | 48.0 g |
| Glycerol | 5.0 g |
| Ethanol | 5.0 g |
| Preserving agent | 1.0 g |

*Polymethyltrifluoropropyldimethylsiloxane

After this foundation was applied to the skin, a makeup with a good ability to absorb the sebum excreted by the skin throughout the day, while remaining uniform, non-shiny and maintaining its initial color, was obtained.

### EXAMPLE 3

A foundation (O/W emulsion) having the composition below was prepared:

| | |
|---|---|
| Polybutylene (INDOPOL ® H100 from Amoco) | 1.0 g |
| Fluorosilicone* (X22-819 from Shin-Etsu) | 2.0 g |
| Isododecane | 19.0 g |
| Sodium lauroyl sarcosinate | 1.0 g |
| Stearic acid | 2.0 g |
| Glyceryl stearate | 2.6 g |
| Glyceryl isostearate | 2.0 g |
| Triethanolamine | 1.0 g |
| Yellow iron oxide | 1.0 g |
| Brown iron oxide | 0.4 g |
| Black iron oxide | 0.1 g |
| Titanium dioxide | 3.9 g |
| Porous silica (SUNSPHERE ® H53 from Asahi Glass) | 5.0 g |
| Coated talc (NOVATECH ® NFP Triple Treated Talc from | 5.0 g |
| Daikin) | |
| Water | 53.0 g |
| Preserving agent | 1.0 g |

After this foundation was applied to the skin, a makeup with a good ability to absorb the sebum excreted by the skin throughout the day, while remaining uniform, non-shiny and maintaining its initial color, was obtained.

### EXAMPLE 4

An anhydrous cast foundation having the composition below was prepared:

| | |
|---|---|
| Polyethylene wax (POLYWAX ® 500 from Bareco) | 4.0 g |
| Polyethylene wax (PERFORMALENE ® 400 polyethylene from | 8.0 g |
| New Phase Technologies) | |
| Cyclohexadimethylsiloxane | 20.0 g |
| Cyclopentadimethylsiloxane | 38.0 g |
| Fluorosilicone* (X22-819 from Shin-Etsu) | 5.0 g |
| Yellow iron oxide | 2.2 g |
| Brown iron oxide | 0.6 g |
| Ultramarine blue | 0.3 g |
| Titanium dioxide | 7.0 g |
| Porous silica (SUNSPHERE ® H53 from Asahi Glass) | 5.0 g |
| Coated sericite (NOVATECH ® NFP Triple Treated Sericite | 2.0 g |
| from Daikin) | |
| Nylon powder (ORGASOL ® 4000 from Atochem) | 7.9 g |

*Polymethyltrifluoropropyldimethylsiloxane

This foundation, when applied to the face, gave a uniform makeup that satisfactorily absorbs sebum throughout the day.

What is claimed is:

1. A cosmetic composition comprising
at least one sebum-absorbing powder;
at least one second powder having a critical surface energy of less than or equal to 24 mN/m; and
a binder liquid fatty phase comprising at least one non-volatile oil with Hansen solubility parameters δ^{L}_{d},δ^{L}ₚ, and δ^{L}ₕ, expressed in J^{1/2}.cm^{-3/2}, such that
Δδ=\sqrt{4(16.4−δ_{d}^{L})²+(0.9−δₚ^{L})²+(4.2−δₕ^{L})²}
wherein Δδ ranges from 2 to 20.

2. The composition according to claim 1, wherein the at least one sebum-absorbing powder has a sebum uptake of greater than or equal to 1 ml/g.

3. The composition according to claim 2, wherein the at least one sebum-absorbing powder has a sebum uptake of greater than or equal to 1.5 ml/g.

4. The composition according to claim 3, wherein the at least one sebum-absorbing powder has a sebum uptake of greater than or equal to 2 ml/g.

5. The composition according to claim 1, wherein the at least one sebum-absorbing powder has a specific surface area of greater than or equal to 300 m²/g.

6. The composition according to claim 5, wherein the at least one sebum-absorbing powder has a specific surface area of greater than or equal to 500 m²/g.

7. The composition according to claim 6, wherein the at least one sebum-absorbing powder has a specific surface area of greater than or equal to 600 m²/g.

8. The composition according to claim 1, wherein the at least one at least one sebum-absorbing powder is chosen from silica powders, acrylic polymer powders, polyamide powders, and silicone elastomer powders.

9. The composition according to claim 1, wherein the at least one sebum-absorbing powder is chosen from polymethyl methacrylate powder, polymethyl methacrylate/ethylene glycol dimethacrylate powder, polyallyl methacrylate/ethylene glycol dimethacrylate powder, and ethylene glycol dimethacrylate/lauryl methacrylate copolymer powder.

10. The composition according to claim 8, wherein the at least one sebum-absorbing powder is a silica powder.

11. The composition according to claim 1, wherein the at least one sebum-absorbing powder is present in an amount ranging from 1% to 98% by weight, relative to the total weight of the composition.

12. The composition according to claim 11, wherein the at least one sebum-absorbing powder is present in an amount ranging from 1% to 80% by weight, relative to the total weight of the composition.

13. The composition according to claim 12, wherein the at least one sebum-absorbing powder is present in an amount ranging from 1% to 60% by weight, relative to the total weight of the composition.

14. The composition according to claim 13, wherein the at least one sebum-absorbing powder is present in an amount ranging from 1% to 35% by weight, relative to the total weight of the composition.

15. The composition according to claim 14, wherein the at least one sebum-absorbing powder is present in an amount ranging from 1% to 15% by weight, relative to the total weight of the composition.

16. The composition according to claim 1, wherein the at least one second powder has a critical surface energy ranging from 10 to 24 mN/m.

17. The composition according to claim 1, wherein the at least one second powder has a critical surface energy of less than or equal to 20 mN/m.

18. The composition according to claim 17, wherein the at least one second powder has a critical surface energy ranging from 10 to 20 mN/m.

19. The composition according to claim 1, wherein the at least one second powder is a powder coated with at least one compound chosen from organofluorine compounds and silicone compounds.

20. The composition according to claim 19, wherein the organofluorine compound is chosen from perfluoroalkyl phosphates and polyhexafluoropropylene oxide.

21. The composition according to claim 20, wherein the perfluoroalkyl phosphate is chosen from those of formula (I):
[CₘF₂ₘ₊₁CₙH₂ₙO]_{y}PO(OM)_{3-y}
wherein
m is an integer ranging from 1 to 21;
n is an integer ranging from 1 to 14;
y is chosen from 1, 2, and 3;
M is chosen from hydrogen, alkali metal cations, ammonium groups, and ammonium groups substituted with 1 to 3 C₁-C₈ alkyl radicals.

22. The composition according to claim 19, wherein the silicone compound is chosen from methicones, dimethicones, polyorganosiloxanes comprising perfluoroalkyl groups, polyorganosiloxanes comprising perfluoropolyether groups, and perfluoroalkylsilanes.

23. The composition according to claim 19, wherein the at least one compound chosen from organofluorine compounds and silicone compounds is present in an amount ranging from 0.01% to 60% by weight, relative to the total weight of the coated powder.

24. The composition according to claim 23, wherein the at least one compound chosen from organofluorine compounds and silicone compounds is present in an amount ranging from 0.05% to 40% by weight, relative to the total weight of the coated powder.

25. The composition according to claim 24, wherein the at least one compound chosen from organofluorine compounds and silicone compounds is present in an amount ranging from 0.1% to 40% by weight, relative to the total weight of the coated powder.

26. The composition according to claim 1, wherein the at least one second powder is present in an amount ranging from 0.1% to 95% by weight, relative to the total weight of the composition.

27. The composition according to claim 26, wherein the at least one second powder is present in an amount ranging from 0.5% to 90% by weight, relative to the total weight of the composition.

28. The composition according to claim 27, wherein the at least one second powder is present in an amount ranging from 5% to 80% by weight, relative to the total weight of the composition.

29. The composition according to claim 1, wherein the at least one non-volatile oil has a viscosity, measured at 25° C., of greater than or equal to 5×10⁻² Pa.s (50 cps).

30. The composition according to claim 29, wherein the at least one non-volatile oil has a viscosity, measured at 25° C., ranging from 5×10⁻² Pa.s to40 Pa.s (40,000 cps).

31. The composition according to claim 30, wherein the at least one non-volatile oil has a viscosity, measured at,25° C., of greater than or equal to 9×10⁻² Pa.s (90 cps).

32. The composition according to claim 31, wherein the at least one non-volatile oil has a viscosity, measured at 25° C., ranging from 9×10⁻² Pa.s to 40 Pa.s.

33. The composition according to claim 1, wherein the at least one non-volatile oil has Hansen solubility parameters δ^{L}_{d}, δ^{L}ₚ, and δ^{L}ₕ, expressed in J^{1/2}.cm^{-3/2}, such that Δδ ranges from 4 to 16.

34. The composition according to claim 33, wherein Δδ ranges from 6 to 16.

35. The composition according to claim 1, wherein the at least one non-volatile oil is such that δ^{L}_{d} is less than or equal to 17.

36. The composition according to claim 35, wherein the at least one non-volatile oil is such that δ^{L}_{d} is less than or equal to 14.

37. The composition according to claim 1, wherein the at least one non-volatile oil is chosen from triisoarachidyl citrate, polyvinylpyrrolidone/hexadecene with a weight-average molecular weight ranging from 5,000 to 9,000, 2-butyloctyl trimellitate, triisostearyl citrate, tridecyl trimellitate, polyglyceryl-2 triisostearate, pentaerythrityl tetrakis(2-ethylhexanoate), triisodecyl trimellitate, diisostearyl malate, tris(2-ethylhexyl) trimellitate, 2-octyldodecanol, octyl hydroxystearate, polybutylene with a weight-average molecular weight ranging from 800 to 1,200, isostearyl lactate, propylene glycol monoisostearate, polyglyceryl-2 diisostearate, castor oil, dipropylene glycol dibenzoate, oxyethylenated (7 EO) glyceryl triacetate, polyglyceryl-3 diisostearate, and the polymethylfluoroalkyldimethylsiloxane of formula (II): 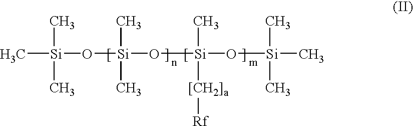
wherein
n is an integer ranging from 5 to 90,
m is an integer ranging from 1 to 150,
a is an integer ranging from 0 to 5 and
Rf is a perfluoroalkyl radical comprising 1 to 8 carbon atoms.

38. The composition according to claim 1, wherein the non-volatile oil is a polymethylfluoroalkyIdimethylsiloxane of formula (Il): 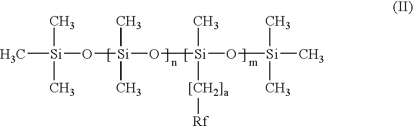
wherein
n is an integer ranging from 5 to 90,
m is an integer ranging from 1 to 150,
a is an integer ranging from 0 to 5 and
Rf is a perfluoroalkyl radical comprising 1 to 8 carbon atoms.

39. The composition according to claim 1, wherein the at least one non-volatile oil is present in an amount ranging from 0.1% to 90% by weight, relative to the total weight of the composition.

40. The composition according to claim 39, wherein the at least one non-volatile oil is present in an amount ranging from 0.1% to 75% by weight, relative to the total weight of the composition.

41. The composition according to claim 40, wherein the at least one non-volatile oil is present in an amount ranging from 1% to 50% by weight, relative to the total weight of the composition.

42. The composition according to claim 41, wherein the at least one non-volatile oil is present in an amount ranging from 1% to 30% by weight, relative to the total weight of the composition.

43. The composition according to claim 1, wherein the liquid fatty phase comprises 100% by weight of the at least one non-volatile oil.

44. The composition according to claim 1, wherein the liquid fatty phase further comprises at least one additional non-volatile oil.

45. The composition according to claim 1, wherein the liquid fatty phase comprises at least one volatile oil.

46. The composition according to claim 45, wherein the at least one volatile oil is chosen from linear or cyclic silicone oils and hydrocarbon-based volatile oils comprising 8 to 16 carbon atoms.

47. The composition according to claim 46, wherein the linear or cyclic silicone oils are chosen from linear or cyclic silicone oils comprising 2 to 7 silicon atoms, wherein the silicon atoms optionally comprise at least one group chosen from alkyl groups comprising 1 to 10 carbon atoms and alkoxy groups comprising 1 to 10 carbon atoms.

48. The composition according to claim 45, wherein the at least one volatile oil is chosen from octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, isododecane, isodecane, and isohexadecane.

49. The composition according to claim 45, wherein the at least one volatile oil is present in an amount ranging from 0.1% to 90% by weight, relative to the total weight of the composition.

50. The composition according to claim 49, wherein the at least one volatile oil is present in an amount ranging from 1% to 50% by weight, relative to the total weight of the composition.

51. The composition according to claim 1, wherein the binder liquid fatty phase comprises an aqueous phase.

52. The composition according to claim 51, wherein the aqueous phase is present in an amount ranging from 0.1% to 60% by weight, relative to the total weight of the composition.

53. The composition according to claim 52, wherein the aqueous phase is present in an amount ranging from 5% to 50% by weight, relative to the total weight of the composition.

54. The composition according to claim 1, further comprising at least one additional powder that is different from the at least one sebum-absorbing powder and the at least one second powder.

55. The composition according to claim 54, wherein the at least one additional powder is chosen from pigments, nacres, fillers, and mixtures thereof.

56. The composition according to claim 54, wherein the at least one additional powder is present in an amount ranging from 0.01% to 98.9% by weight, relative to the total weight of the composition.

57. The composition according to claim 56, wherein the at least one additional powder is present in an amount ranging from 0. 1% to 85% by weight, relative to the total weight of the composition.

58. The composition according to claim 56, wherein the at least one additional powder is present in an amount ranging from 1% to 70% by weight, relative to the total weight of the composition.

59. The composition according to claim 1, further comprising at least one cosmetic ingredient chosen from antioxidants, fragrances, preserving agents, neutralizers, surfactants, waxes, gelling agents, film-forming polymers, thickeners, sunscreens, vitamins, moisturizers, self-tanning compounds, antiwrinkle active agents, and mixtures thereof.

60. The composition according to claim 1, wherein the composition is in a form chosen from compact powders, cast powders, free powders, gels, water-in-oil emulsions, oil-in-water emulsions, multiple emulsions, milks, and pastes.

61. The composition according to claim 1, wherein the composition is anhydrous.

62. The composition according to claim 1, wherein the composition is in a form chosen from foundations, rouges, eyeshadows, concealer products, face and body powders, and body makeup products.

63. A non-therapeutic cosmetic process for making up and/or caring for keratin materials, comprising applying to the keratin materials a composition comprising
at least one sebum-absorbing powder;
at least one second powder having a critical surface energy of less than or equal to 24 mN/m; and
a binder liquid fatty phase comprising at least one non-volatile oil with Hansen solubility parameters δ^{L}_{d}, δ^{L}ₚ, and δ^{L}ₕ, expressed in J^{1/2}.cm^{-3/2}, such that
Δδ=\sqrt{4(16.4−δ_{d}^{L})²+(0.9−δₚ^{L})²+(4.2−δₕ^{L})²}
wherein Δδ ranges from 2 to 20.

64. A process for obtaining a non-shiny, uniform, transfer-resistant and/or rub-resistant makeup result on the skin and/or a makeup result that shows maintenance of the initial color over time, comprising applying to keratin materials a cosmetic composition comprising
at least one sebum-absorbing powder;
at least one second powder having a critical surface energy of less than or equal to 24 mN/m; and
a binder liquid fatty phase comprising at least one non-volatile oil with Hansen solubility parameters δ^{L}_{d}, δ^{L}ₚ, and δ^{L}ₕ, expressed in J^{1/2}.cm^{-3/2}, such that
Δδ=\sqrt{4(16.4−δ_{d}^{L})²+(0.9−δₚ^{L})²+(4.2−δₕ^{L})²}
wherein Δδ ranges from 2 to 20.
